(19) United States
(12) Patent Application Publication
(10) Pub. No.: US 2004/0204703 A1
(43) Pub. Date: Oct. 14, 2004

(54) Method and system for treating presbyopia

(76) Inventors: George W. Rozakis, Lakewood, OH (US); Guillermo Avalos, Guadalajara (MX)

Correspondence Address: SAND & SEBOLT, AEGIS TOWER, SUITE 1100, 4940 MUNSON STREET, NW, CANTON, OH, 44718-3615, US

(21) Appl. No.: 10/833,221

(22) Filed: Apr. 27, 2004

Related U.S. Application Data
(62) Division of application No. 09/752,273, filed on Dec. 29, 2000, now abandoned.
(60) Provisional application No. 60/173,448, filed on Dec. 29, 1999.

(51) Int. Cl. A61B 18/20

(52) U.S. Cl. 606/5

(57) ABSTRACT

A system for correcting presbyopia provides a laser adapted to sculpt the cornea in two concentric zones. The first larger zone is treated with a positive diopter correction to allow eye to focus on near objects. A second smaller zone is then treated with a negative diopter correction to allow the cornea to focus on far objects. In one embodiment, the system directs the laser in a series of collapsing crescents to sculpt the zones.

### CROSS REFERENCE TO RELATED APPLICATIONS

This application is a divisional application claiming priority from U.S. patent application Ser. No. 09/752,273 filed Dec. 29, 2000; which claims priority from U.S. Provisional Patent application serial No. 60/173,448 filed Dec. 29, 1999; the disclosures of both are incorporated herein by reference.

### BACKGROUND OF THE INVENTION

1. Technical Field

The present invention generally relates to a method and system for performing surgery on the cornea to correct vision defects. More particularly, the present invention relates to a system and method for treating presbyopia by altering the light transmitting properties of the cornea. Specifically, the present invention relates to a system and method for altering the shape of the cornea to have two concentric vision zones that allow a presbyopic patient to focus on near items and far items.

2. Background Information

The human eye transforms light waves into electrical impulses that are interpreted by the brain. Light waves enter the eye through a transparent cornea. The waves then pass through the aqueous humor and through the pupil. The iris expands or constricts the pupil to allow the proper amount of light to pass to the lens. The light waves then pass through the crystalline lens. The lens is focused with the ciliary muscle so that the light waves are properly focused on the retina. The retina includes light sensitive cells (rods and cones) that transform light waves into electrical impulses. The electrical impulses are transmitted through the optic nerve to the brain. The visual cortex at the back of the brain reconstructs the impulses into an image.

Presbyopia is a natural condition causing the eye to lose its ability to focus on near objects. Over the past several years, presbyopia has been thought to occur due to the loss of flexibility of the crystalline lens in the eye. Presbyopia has traditionally been treated with reading glasses or bifocals that allow the patient to focus on near objects. Surgical correction procedures have not been attempted because of the understanding that presbyopia occurs because the lens has hardened.

In recent years, another theory has developed stating that presbyopia is caused by the growth of the lens causing the ligaments to loosen so that they can no longer exert tension on the lens. A surgical correction method has been developed based on this theory wherein tension is re-introduced to the ligaments supporting the lens by inserting implants.

It is desired in the art to provide a surgical treatment for presbyopia where implants are not required. It is desirable that such a method be as successful and as relatively simple as the widely-used laser refractive surgery methods now known in the art.

### SUMMARY OF THE INVENTION

The invention provides a system and a method for treating presbyopia by altering the light transmitting properties of the cornea. The invention provides a method wherein two concentric areas of the cornea are shaped to allow the patient to focus on both near and far objects. The larger area is treated to focus on close objects while the inner smaller area is treated to focus on far objects. The method of the invention may be performed on a system that creates collapsing crescent-shaped eroding laser areas.

The invention allows refractive errors of the eye to be corrected while also correcting the presbyopic condition of the eye.

The invention may also be used to treat myopia.

### BRIEF DESCRIPTION OF THE DRAWINGS

FIG. 1 is a cross-sectional view of a typical human eye;

FIG. 2 is a front elevational view of the cornea of the eye;

FIG. 3 is a sectional view taken along line 3-3 of FIG. 2;

FIG. 4A is a view similar to FIG. 2 showing the first stage of a collapsing crescent laser beam applied to the left side of the cornea;

FIG. 4B is a view similar to FIG. 4A showing an intermediate stage;

FIG. 4C is a view similar to FIG. 4A showing a final stage of the collapsing crescent laser beam;

FIG. 5A is a view similar to FIG. 4A showing an initial stage of the collapsing crescent laser beam applied to the right side of the cornea;

FIG. 5B is a view similar to FIG. 5A showing an intermediate stage;

FIG. 5C is a view similar to FIG. 5A showing a final stage of the collapsing crescent laser beam;

FIG. 6A is a view similar to FIG. 4A showing an initial stage of the collapsing crescent laser beam applied to the top half of the cornea;

FIG. 6B is a view similar to FIG. 6A showing an intermediate stage;

FIG. 6C is a view similar to FIG. 6A showing a final stage of the collapsing crescent laser beam;

FIG. 7A is a view similar to FIG. 4A showing an initial stage of the collapsing crescent laser beam applied to the bottom half of the cornea;

FIG. 7B is a view similar to FIG. 7A showing an intermediate stage;

FIG. 7C is a view similar to FIG. 7A showing a final stage of a collapsing crescent laser beam;

FIG. 8 is a sectional view similar to FIG. 3 showing the cornea after the laser treatment of FIGS. 4A-7C;

FIG. 9 is a view similar to FIG. 2 showing the cornea of FIG. 8;

FIG. 10 is a sectional view taken along line 10-10 of FIG. 9;

FIG. 11 is a sectional view similar to FIG. 3 showing the resulting shape of the cornea after the treatment of the present invention; and

FIG. 12 is a schematic of the apparatus used to perform the method of the present invention.

Similar numbers refer to similar elements throughout the specification.

### DESCRIPTION OF THE PREFERRED EMBODIMENTS

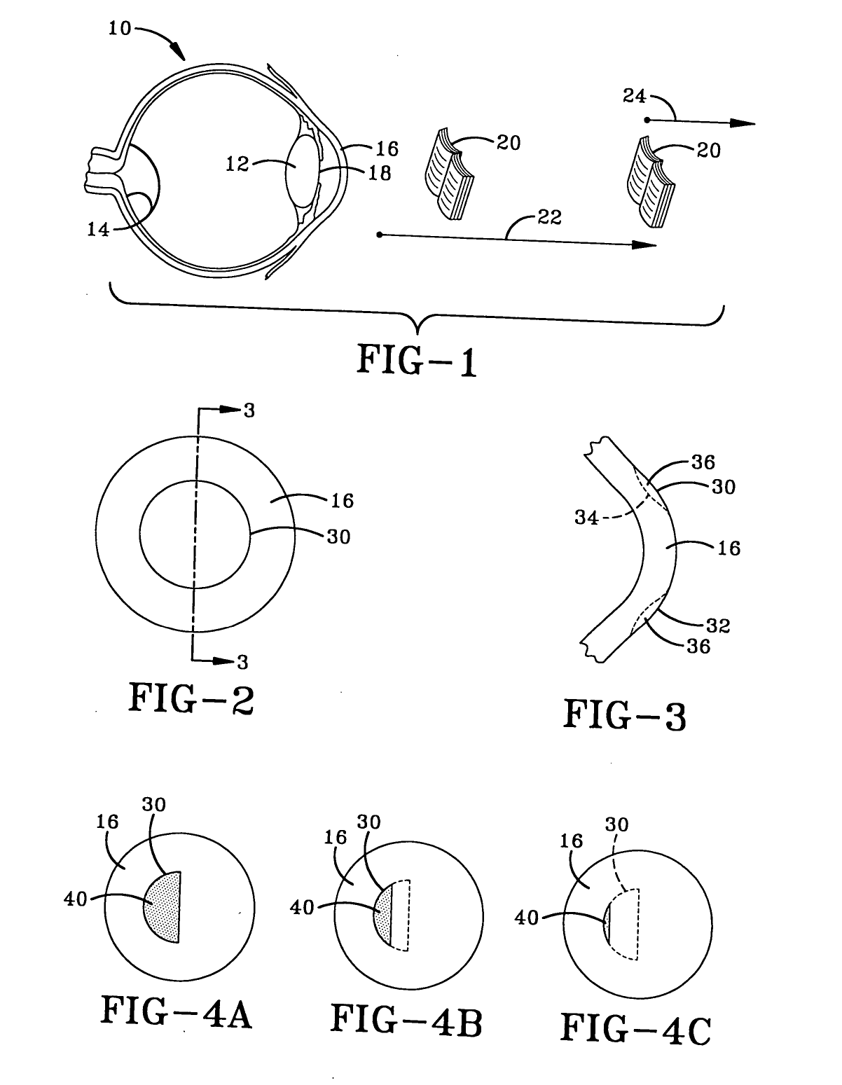

Presbyopia is a condition that affects most human eyes as the eye ages. A typical human eye is depicted in FIG. 1 and is indicated generally by the numeral 10. Eye 10 includes a lens 12 that focuses light waves on the retina 14. The light waves enter the eye through the cornea 16 and pass through the pupil 18 to lens 12. When eye 10 is young, lens 12 can focus light waves on retina 14 from objects that are relatively close to cornea 16. For instance, the young eye may properly focus on the text in a book 20 that is as close as two inches from cornea 16. As the eye ages, lens 12 loses its ability to focus on near objects and eventually book 20 must be held ten to twenty inches away from cornea 16 in order for lens 12 to focus on the text. The young eye thus has an ability to focus in the range indicated by the arrow 22. The old eye 10 can only focus in the range indicated by the numeral 24. This condition is known as presbyopia and has traditionally been treated by supplying reading glasses to supplement lens 12 allowing eye 10 to focus back into range 22. One problem with reading glasses is that the wearer of reading glasses loses his ability to focus on far objects while wearing the reading glasses. The method of the present invention alters the shape of cornea 16 allowing eye 10 to focus on near objects as well as far objects.

The method of the present invention is performed by first determining the shape and focusing ability of eye 10. Various methods for determining these factors are known in the art and any of the variety of methods may be used to gather this information. For instance, the first step may be to use a phoropter to measure the refractive state of eye 10. The person performing the method may optionally determine the topography of cornea 16 with a topographer. The pachymetry or corneal thickness may also be optionally measured.

Performing these steps will determine if eye 10 is presbyopic piano, presbyopic with spherical hyperopia, or presbyopic with spherical myopia. It may also be determined that any of these three conditions is combined with astigmatism. When eye 10 has astigmatism, the astigmatism is first treated using a LASIK algorithm to correct for the astigmatism. The patient then must wait for eye 10 to heal (approximately two months) before the method of the present invention is performed.

The doctor performing the method next determines an appropriate level of correction that must be applied to cornea 16 to allow eye 10 to focus on objects in range 22. In the first embodiment discussed below, eye 10 is presbyopic but piano meaning that no correction is needed to achieve 20/20 vision other than the correction for the presbyopic condition. In the preferred embodiment of the invention, two diopters of correction will be used to correct the presbyopic condition. In other embodiments, a different correction may be used. The doctor then selects a central area 30 (FIG. 2) on cornea 16 to perform the initial treatment. In the preferred embodiment, area 30 is a 6 mm optical zone.

The patient's eye is first anesthetized as is known in the art. The doctor cuts a flap in cornea 16 as is known in the art so that the actual laser treatment on area 30 is performed under the outer surface of cornea 16 in the corneal stroma. In other embodiments, the doctor may remove a disk from the cornea. The disk is replaced after the cornea is sculpted. The FIG. 3 shows the topography of cornea 16 before the method of the present invention is applied to cornea 16 as identified with solid line 32. The initial two diopter treatment will change the shape of cornea 16 to that indicated by dashed line 34. The two diopter treatment steepens the curvature in area 30 so that eye 10 can focus on close objects 20 in range 22.

The initial treatment on cornea 16 is performed by eroding area 36 with a laser beam. The boundaries and definition of area 36 is determined using a difference of sphere algorithm or other suitable algorithms known in the art. The resulting area 36 is mathematically defined is a way that a laser can be controlled to remove area 36. The apparatus to sculpt a cornea with a laser beam is known in the art and is preferably an excimer laser. One example of an apparatus useful for performing this method is disclosed in U.S. Pat. No. 5,642,287, the disclosures of which are incorporated herein by reference. The preferred treatment for removing area 36 is to sculpt cornea 16 with four sequential collapsing crescent-shaped laser beams. In other embodiments of the invention, area 36 may be removed using different known methods such as a scanning spot or an erodible mask. The collapsing crescent-shaped laser beams are applied to area 30 in four steps. FIGS. 4A-7C show the application of laser beam 40 to area 30 on cornea 16. The specific sequence of FIGS. 4, 5, 6, and 7 is irrelevant to the present invention. The order provided in the drawings is provided as an example but it is understood that the doctor may start on the right half, the upper half, or the lower half instead of the left half as shown in the drawings.

The treatment begins with a half circle-shaped laser applied to the left half of area 30 as depicted in FIG. 4A. The intensity of laser 40 and the amount of time it is applied to area 30 are determined by a controller that analyzes area 36 and determines the time and intensity required for laser 40 to remove area 36. Laser 40 then collapses from the shape shown in FIG. 4A towards the shape shown in FIG. 4C. It is thus understood that the size of laser 40 gradually collapses until it disappears in the direction from the center of area 30 to the edge of area 30.

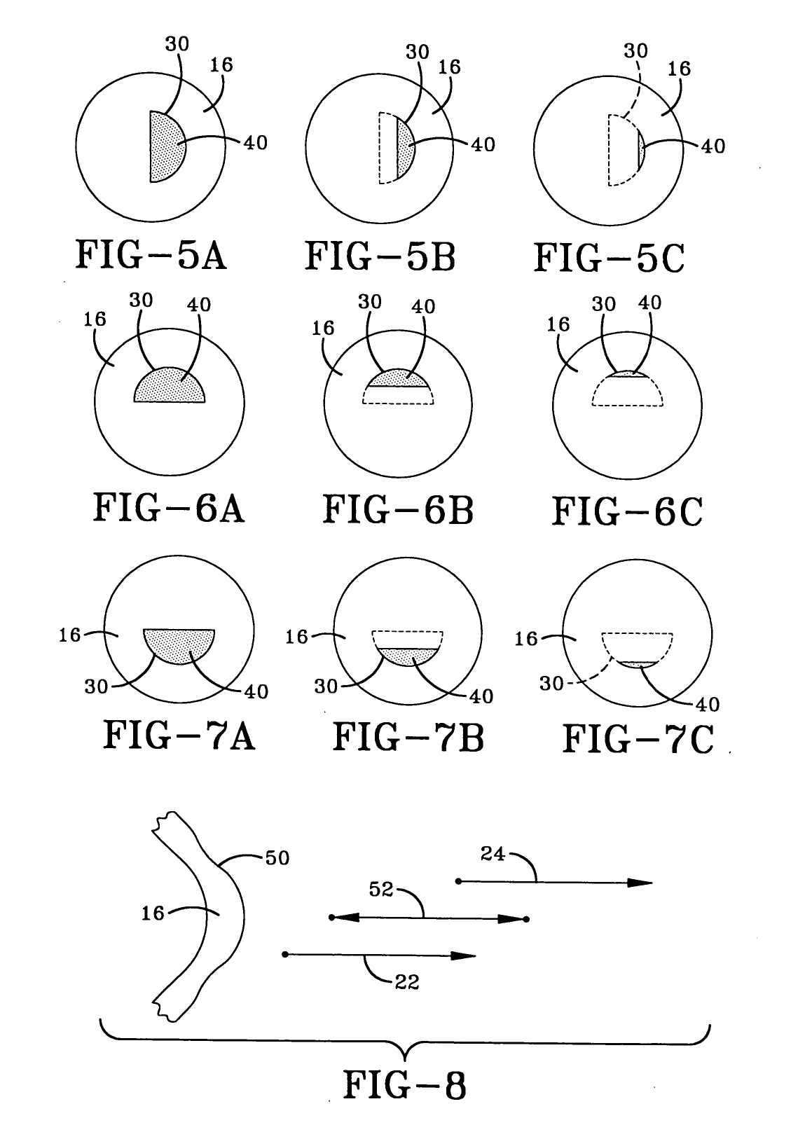

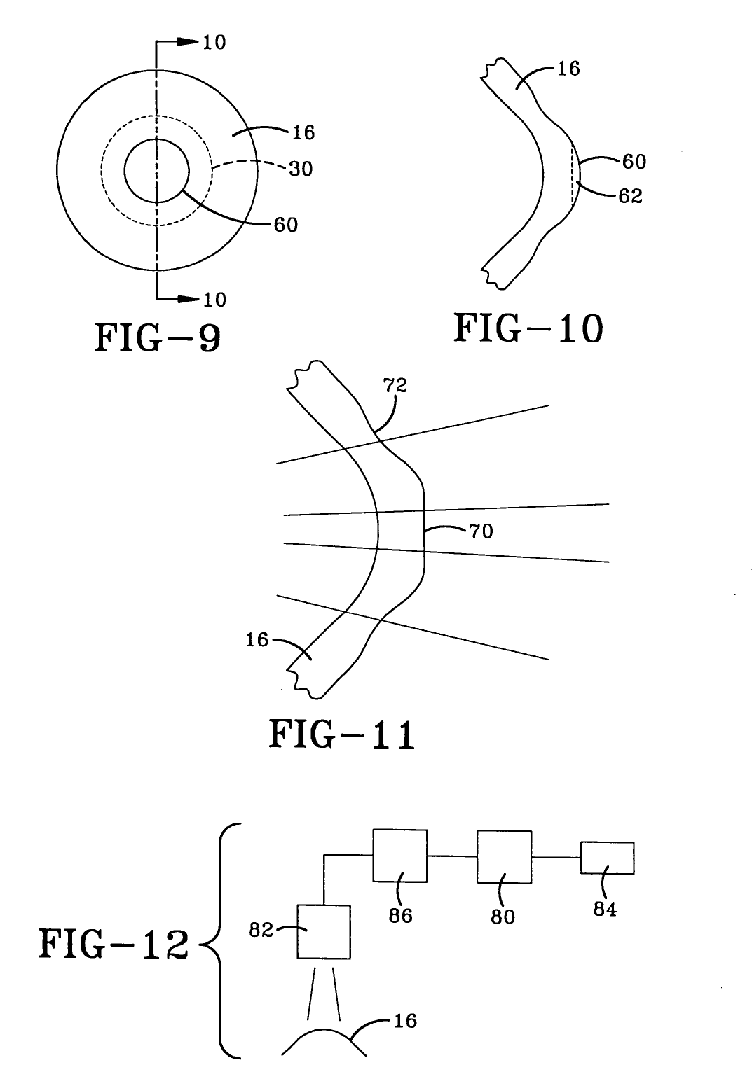

The doctor then treats the right side of area 30 as depicted in FIGS. 5A-5C. Once the left and right sides of area 30 are treated, the doctor changes the laser configuration and treats the top and bottom sections of area 30 as depicted in FIGS. 6A-6C and 7A-7C.

The resulting cornea is depicted sectionally in FIG. 8. Treated cornea 16 now has a steepened section 50 that allows eye 10 to focus in range 52 that includes most of range 22, some of range 24, but excludes the far end of range 24. The cornea of FIG. 8 is thus myopic because it cannot focus on objects far away from cornea 16. However, the presbyopic eye can now focus on near objects in range 52.

Having created the cornea FIG. 8, the doctor now selects a smaller area 60 that is substantially concentric with area 30. In the preferred embodiment of the invention, area 60 is a 4 mm optical zone. The doctor applies a treatment to area 60 that allows area 60 to focus on objects far away thus treating the myopic condition. This treatment is a spherical diopter correction equal to, but negative of, the positive correction applied to area 30. In this embodiment, a negative 2 diopter correction is performed in area 60.

The negative correction applied to area 60 will remove area 62 of cornea 16 to provide a central area of eye 10 that will be able to focus on far objects. Material 62 is defined and removed using one of the same methods described above.

The resulting cornea is depicted in FIG. 11. Cornea 16 has a central area 70 that is configured to allow eye 10 to focus on far objects and a ring-shaped area 72 that allows eye 10 to focus on near objects. Having undergone this treatment, the presbyopic patient can now focus on near and far objects without the use of implants or reading glasses.

As noted above, the initial examination of eye 10 may determine that eye 10 is hyperopic or myopic. When eye 10 is hyperopic, the correction described above is altered to also correct for the hyperopia. For instance, zone 30 is initially treated with a two diopter correction for the presbyopia along with a correction for the hyperopia. In one example where the hyperopia is plus three diopters, the correction first applied to area 30 is plus three diopters for the hyperopia and plus two diopters for the presbyopia resulting in a positive five diopter spherical correction in zone 30. Zone 60 is then treated with a negative two spherical diopter correction resulting in a correction of the hyperopia in addition to the presbyopia.

When eye 10 is myopic, the initial correction to area 30 includes a combination of the presbyopic correction with a myopic correction. For example, when eye 10 is negative 3 diopters myopic, the presbyopia correction to area 30 is a negative 1 diopter correction in area 30 determined by combining a negative 3 diopter spherical correction with a positive 2 diopter spherical correction. Area 60 is then treated with a negative 2 spherical diopter correction.

After the sculpting operations are performed, a topography of the sculpted eye may be taken to show the difference of curvature in zones 60 and 30. The doctor then examines the patient's distance visual acuity and checks the patient's near visual acuity. If further errors are found, further sculpting is performed.

The apparatus used to perform this method includes a computer 80 that is capable of controlling a laser 82 used to ablate cornea 16. Computer 80 is capable of controlling laser 82 to define and remove areas 36 and 62. Computer 80 may include an input device 84, such as a keyboard, that allows information about eye 10 to be placed in the memory of computer 80. A controller 86 may also be provided that allows computer 80 to communicate with laser 82.

The method and apparatus described above may also be used to treat a myopic cornea. In this treatment, distance is treated in the center and the periphery is treated less to create a solution for the myopic eye. For instance, a 4 millimeter center area may be treated to view distant objects. The peripheral region (outside diameter of 6 millimeters) is treated with a corresponding treatment to allow the eye to see near objects. The 6 millimeter zone is treated first by creating a curvature allowing the 6 millimeter zone to see near objects. A 4 millimeter zone is then treated to flatten the 4 millimeter zone so that it may focus on far objects. The combined zones allow the cornea to focus on near and far objects.

In the foregoing description, certain terms have been used for brevity, clearness, and understanding. No unnecessary limitations are to be implied therefrom beyond the requirement of the prior art because such terms are used for descriptive purposes and are intended to be broadly construed.

Moreover, the description and illustration of the invention is an example and the invention is not limited to the exact details shown or described.

1. A method for treating the cornea of an eye for correcting presbyopia, the method comprising the steps of:
(a) selecting a first zone of the cornea having a first diameter;
(b) treating the first zone with a positive diopter correction;
(c) selecting a second zone of the cornea having a second diameter, the second diameter being smaller than the first diameter; and
(d) treating the second zone with a negative diopter correction.

2. The method of claim 1, wherein the second zone is concentric with the first zone.

3. The method of claim 1, wherein the first diameter is 6 millimeters.

4. The method of claim 3, wherein the second diameter is 4 millimeters.

5. The method of claim 4, wherein the diopter power is 2.

6. The method of claim 5, wherein step (b) is performed by using a laser beam in the shape of a collapsing crescent.

7. The method of claim 6, wherein step (b) is performed by sequentially sculpting four areas with collapsing crescents.

8. The method of claim 6, wherein step (b) is performed by sequentially sculpting four areas with collapsing crescents wherein the four areas are disposed 90 degrees with respect to each other.

9. The method of claim 6, wherein step (d) is performed by using a laser beam in the shape of a collapsing crescent.

10. The method of claim 9, wherein step (d) is performed by sequentially sculpting four areas with collapsing crescents.

11. The method of claim 9, wherein step (d) is performed by sequentially sculpting four areas with collapsing crescents wherein the four areas are disposed 90 degrees with respect to each other.

12. The method of claim 1, wherein step (b) is performed by using a scanning spot laser.

13. The method of claim 12, wherein step (d) is performed by using a scanning spot laser.

14. The method of claim 1, wherein steps (b) and (d) use the same diopter power.

15. The method of claim 1, further comprising the step of determining the shape and focusing ability of the eye before step (a).

16. The method of claim 15, further comprising the steps of:
anesthetizing a patient;
resecting at least a portion of a cornea of an eye of the patient to expose a corneal stroma;
performing step (b) on the corneal stroma;
performing step (d) on the corneal stroma after step (b) is complete such that step (d) is being performed on an area that has already been treated; and repositioning the portion of the cornea onto the eye wherein a central corneal curvature change is induced to thereby correct presbyopia in the patient.

17. The method of claim 16, wherein in the resecting step, the cornea is resected such that a portion of the cornea remains intact, and the cornea is folded back to expose the corneal stroma.

18. The method of claim 16, wherein in the resecting step, the cornea is resected such that a disk of the cornea is removed from the eye, to thereby expose the corneal stroma.

19. A method for treating the cornea of an eye for correcting presbyopia, the method comprising the steps of:
(a) resecting portion of a cornea to expose a first zone of the corneal stroma;
(b) treating the first zone with a positive diopter correction to define a treated first zone;
(c) selecting a second zone inside the treated first zone of the cornea; the second zone being entirely inside the first treated zone;
(d) treating the second zone with a negative diopter correction; and
(e) repositioning the portion of the cornea onto the eye wherein a central corneal curvature change is induced to thereby correct presbyopia.

20. The method of claim 19, wherein steps (b) and (d) use the same diopter power.

21. The method of claim 19, wherein steps (b) and (d) use different diopter powers.
